(19) United States
(12) Patent Application Publication
(10) Pub. No.: US 2011/0160766 A1
(43) Pub. Date: Jun. 30, 2011

(54) Medical Affixation Device

(76) Inventors: Ronald D. Hendren, Hopkins, MN (US); Douglas A. Becker, Minnetonka, MN (US)

(21) Appl. No.: 13/044,835

(22) Filed: Mar. 10, 2011

Related U.S. Application Data
(62) Division of application No. 11/265,613, filed on Nov. 2, 2005.

(51) Int. Cl. A61B 17/04

(52) U.S. Cl. 606/232

(57) ABSTRACT

A medical affixation device for affixing a first body tissue portion to a second body tissue portion in vivo includes a main body portion having a first surface operably oriented in facing relationship with a first body tissue, wherein the first surface has a non-planar contour forming one or more body tissue contact portions and one or more non-contacting portion. The body tissue contact portions have an effective tissue contact surface area that is sufficiently large to prevent the body tissue contact portions from penetrating the first body tissue when the device is fully installed. The device further includes one or more prongs extending from the main body portion, and which are configured to penetrate the first body tissue upon installation of the device.

### CROSS REFERENCE TO RELATED APPLICATIONS

This application is a divisional of U.S. patent application Ser. No. 11/265,613, entitled “Medical Affixation Device”, filed Nov. 2, 2005, the content of which being incorporated herein in its entirety.

### FIELD OF THE INVENTION

The present invention relates to medical affixation devices generally, and more particularly to implantable devices such as medical staples and plates that minimize damage to soft tissue when utilized in vivo. In addition, the present invention relates to suture anchoring devices that provide an efficient platform to which tissue may be securely tethered.

### BACKGROUND OF THE INVENTION

Medical affixation devices of various forms have long been used for stabilizing and/or securing body tissues in vivo. Common examples of such medical affixation devices include staples, plates, and pins that are manufactured from biocompatible materials so that such devices may be implanted within the body either temporarily or permanently without adverse reaction from the patient's immune system.

In many applications, the medical affixation devices are affixed to a solid structure within the body, such as a bone. In doing so, soft tissue adjacent to the respective bone, such as tendons, ligaments, muscular tissue, or other connective or protective tissue, is clamped between the affixation device and the bone. Since the affixation devices are typically firmly secured to the bone, the soft tissue disposed intermediately between the affixation device and the bone is often times tightly pinched therebetween. Such pressure upon the soft tissue tends to constrict or block altogether capillary vessels coursing through the soft tissue. As a result, nutrients and oxygen are prevented from supplying the pinched soft tissue, thereby resulting in damage or destruction to the pinched soft tissue.

It has been found that such soft tissue damage can lengthen surgical recovery time and can require physical therapy to re-establish appropriate functionality originally provided by the damaged tissue. In some cases, the functionality provided by the damaged tissue may be permanently lost.

In other applications, it is desirable to secure or re-attach separate tissue elements to one another in vivo. An example of such an operation is in the securement of a bone chip to the respective bone from which the chip originated. Typically, medical “pins” in the form of bolts are inserted through bores in the respective bone and bone chip to draw the pieces together. Such a methodology, however, requires a relatively large bore diameter, and can lead to soft tissue damage, as described above. Other methodologies currently employed to secure such tissue portions to one another are relatively time-consuming and complex.

It is therefore a principal object of the present invention to provide a medical affixation device useful in body tissue securement applications in vivo, while minimizing detrimental effects on soft tissue pressed between the affixation device and the anchoring tissue.

It is a further object of the present invention to provide an affixation device having a suture anchoring mechanism integrated therewith for efficiently grasping suture utilized in securing two distinct body tissue portions to one another.

### SUMMARY OF THE INVENTION

By means of the present invention, damage to soft tissue incurred through in vivo affixation procedures may be substantially reduced. To do so, the medical affixation device of the present invention focuses adherence impact at body tissue contact portions, which represent only a fraction of the total surface area of the main body portion of the affixation device. As a result, the remaining non-contacting portions either remain spaced from the damage-susceptible soft body tissue, or come into contact with such tissue at a force sufficiently low to prevent damage thereto.

The present invention further provides for a mechanism for anchoring sutures in vivo. In particular, the medical affixation device of the present invention preferably utilizes mechanisms for securely engaging with a suture or other tie device, such that a body tissue to which the suture is affixed may be anchored by the affixation device installed in a separate body tissue, such as a bone.

In a particular embodiment, the medical affixation device includes a main body portion having a first surface operably oriented in facing relationship with a first body tissue, with the first surface having a non-planar contour forming one or more body tissue contact portions and one or more non-contacting portions. The body tissue contact portions preferably include an effective tissue contact surface area that is sufficiently large so as to prevent the body tissue contact portions from penetrating the first body tissue when the device is fully installed. The affixation device further includes one or more prongs extending from the main body portion, and are configured to penetrate the first body tissue upon installation of the device.

In another embodiment, the medical affixation device includes a main body portion having a first surface and a generally opposed second surface, and one or more apertures extending therethrough between the first and second surfaces, wherein the first surface is operably oriented in facing relationship with a first body tissue. The affixation device further includes one or more prongs extending from the main body portion that are configured to penetrate said first body tissue upon installation of the device. A suture anchoring mechanism is further provided in the affixation device, and is selectively and retainably disposed in a respective one of the one or more apertures.

The present invention further provides a method for affixing a first body tissue portion to a second body tissue portion in vivo by first providing an affixation device having a main body portion which includes a first surface operably oriented in facing relationship with the first body tissue portion, wherein the first surface has a non-planar contour forming one or more body tissue contact portions and one or more non-contacting portions. The body tissue contact portions preferably have an effective tissue contact surface area that is sufficiently large to prevent the body tissue contact portions from penetrating the first body tissue portion when the device is fully installed. The affixation device further includes one or more prongs extending from the main body portion, which prongs are configured to penetrate the first body tissue portion upon installation of the device. The method is completed by installing the affixation device by inserting the one or more prongs through the first body tissue portion and into the second body tissue portion to an extent sufficient to cause the one or more body tissue contact portions to operably press at least a portion of the first body tissue portion into intimate contact with the second body tissue portion.

A further method contemplated by the present invention includes providing an affixation device having a main body portion having a first surface and a generally opposed second surface, and one or more apertures extending therethrough between the first and second surfaces. The affixation device preferably further includes one or more prongs extending from the main body portion, and a suture anchoring mechanism. The first end of an elongated suture is preferably secured to the second body portion, and a second end of the suture is passed through a respective one of the one or more apertures, and into engaging relationship with the suture anchoring mechanism. The affixation device is installed by inserting the one or prongs into the body tissue portion, and positioning the suture anchoring mechanism in engaging relationship with the main body portion.

### BRIEF DESCRIPTION OF THE DRAWINGS

FIG. 1 is a perspective view of a medical affixation device of the present invention;

FIG. 2 is a perspective view of the medical affixation device illustrated in FIG. 1;

FIG. 3 is a is a perspective view of a medical affixation device of the present invention;

FIG. 4 is a perspective view of a medical affixation device of the present invention;

FIG. 5 is an isolation perspective view of a portion of the medical affixation device illustrated in FIG. 4;

FIG. 6 is a top view of the portion of the medical affixation device illustrated in FIG. 5;

FIG. 7 is a bottom view of the portion of the medical affixation device illustrated in FIGS. 5 and 6;

FIG. 8 is a perspective view of a medical affixation device of the present invention;

FIG. 9 is a perspective view of a medical affixation device of the present invention;

FIG. 10 is a perspective view of a medical affixation device of the present invention; and

FIG. 11 is a perspective view of a medical affixation device of the present invention.

### DETAILED DESCRIPTION OF THE PREFERRED EMBODIMENTS

The objects and advantages enumerated above together with other objects, features, and advances represented by the present invention will now be presented in terms of detailed embodiments described with reference to the attached drawing figures which are intended to be representative of various possible configurations of the invention. Other embodiments and aspects of the invention are recognized as being within the grasp of those having ordinary skill in the art.

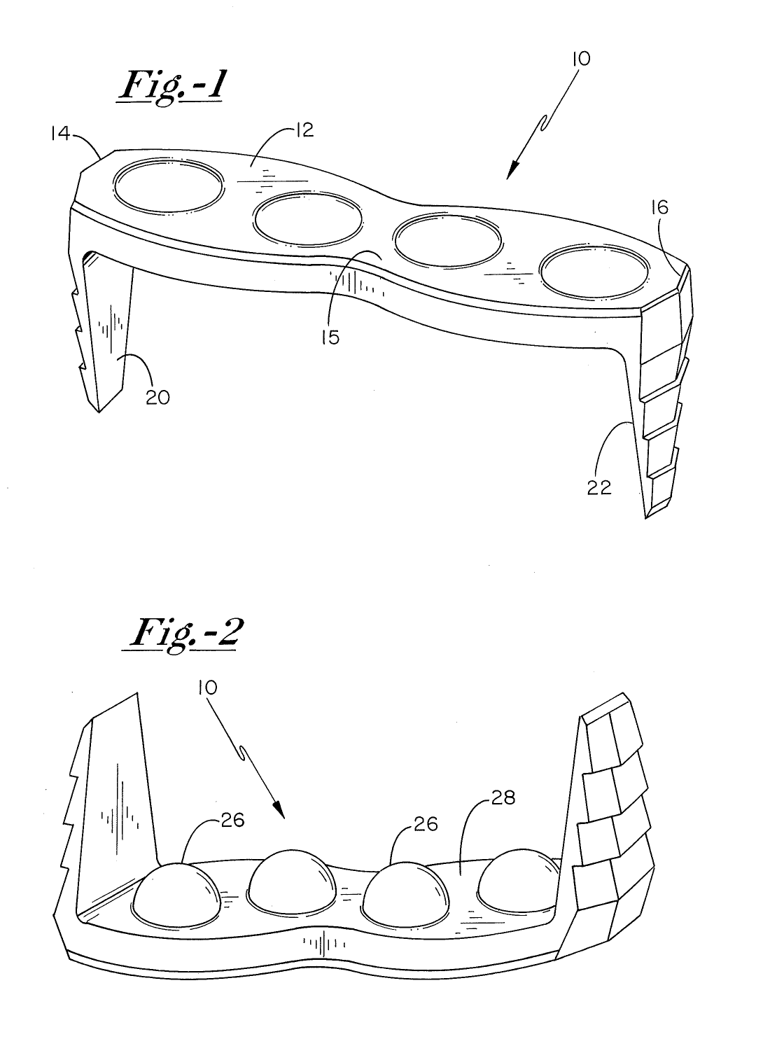

With reference now to the drawing figures, and first to FIG. 1, a medical affixation device 10 includes a main body portion 12 having first and second opposed ends 14, 16, from which first and second prongs 20, 22 extend. In preferred embodiments, affixation device 10 takes the form of a medical staple, although a variety of other configurations, such as medical plates, pins, and the like are contemplated as being within the scope of the present invention.

As illustrated in FIGS. 1 and 2, first surface 13 of main body portion 12 has a non-planar contour so as to form body tissue contact portions 26 and non-contacting portions 28 thereof. Body tissue contact portions 26 of affixation device 10 are preferably configured as substantially hemispherical protrusions extending from first surface 13 of main body portion 12 in a direction substantially parallel to first and second prongs 20, 22 extending from main body portion 12. In such a manner, first surface 13 is operably positioned in facing relationship with a body tissue being impacted by affixation device 10.

In the embodiments illustrated in FIGS. 1 and 2, body tissue contact portions 26 are formed by depressing portions of second surface 15 of main body portion 12. Body tissue contact portions 26, however, may instead be formed as, for example, portions of first surface 13 that are not depressed, separate structures molded to first surface 13, or the lowest regions of a non-linear or non-planar main body portion 12. In particular, an important aspect of the present invention is to provide first surface 13 of main body portion 12 with a non-planar contour, such that certain portions of first surface 13 are lower than other portions thereof. These relatively lower portions form the surface area of first surface 13 that supports the remainder of first surface 13 in a spaced-apart relationship with the soft body tissue upon implantation of affixation device 10.

In order to enable the tissue-saving characteristic of the present invention, body tissue contact portions 26 preferably have a total effective tissue contact surface area that is sufficiently large to prevent body tissue contact portions 26 from penetrating the soft body tissue when affixation device 10 is fully installed. The effective tissue contact surface area of body tissue contact portions 26 is calculated as the total surface area of first surface 13 coming into contact with the soft tissue without penetration into the soft tissue by body tissue contact portions 26. Such contact surface area is therefore dependent upon the force employed in reaching for installation of affixation device 10 in vivo. In typical applications, the effective tissue contact surface area for first surface 13 is at least about 10% of the total surface area of first surface 13, and more preferably between bout 15-80% of the total surface area of that first surface 13. In embodiments including more than one body tissue contact portions 26, such effective tissue contact surface area may be equally or non-equally divided there amongst.

In some embodiments of the present invention, affixation device 10 includes one or more piercing members 32 extending from at least one of body tissue contact portions 26. Such tissue piercing members 32 are preferably incorporated with affixation device 10 so as to enhance the grasping effectiveness of first surface 13 to the body tissue to which affixation device 10 is moored. Accordingly, tissue piercing members 32 are preferably configured to operably penetrate through the soft tissue and into, for example, the bone, to, along with first and second prongs 20, 22, anchor affixation device 10 thereto.

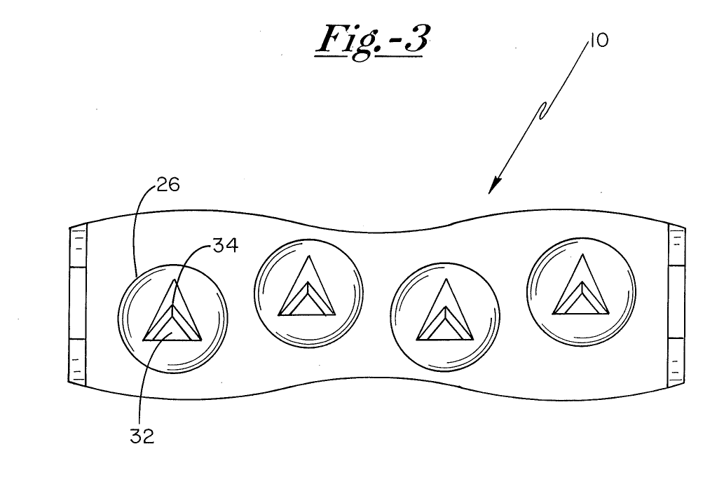

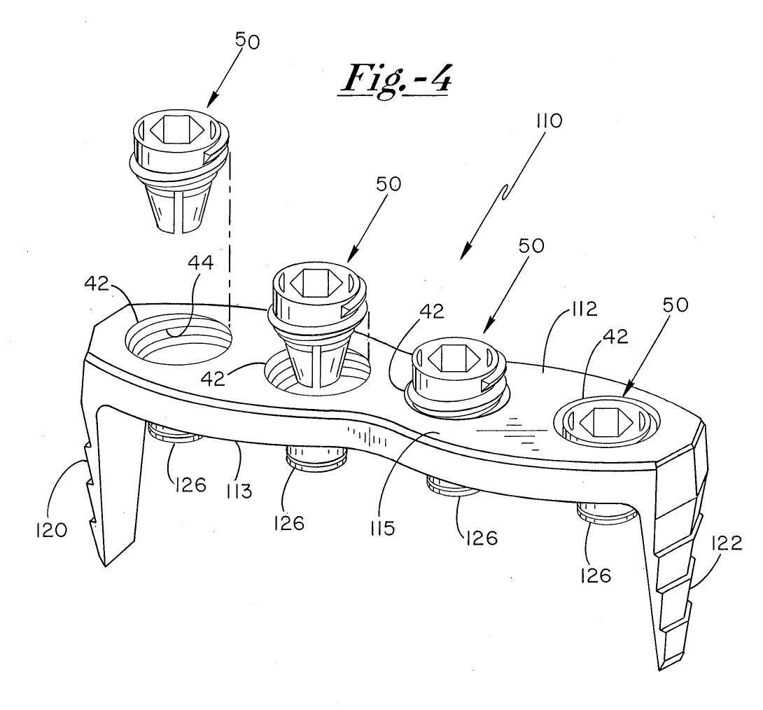

Tissue piercing members 32 preferably extend from first surface 13, and more preferably from body tissue contact portions 26 in a direction substantially parallel to first and second prongs 20, 22, in that such tissue piercing members 32 are operably oriented toward the body tissue to which affixation device 10 is to be installed. In the embodiment illustrated in FIG. 3, tissue piercing members 32 are formed as partial cut-outs of respective ones of body tissue contact portions 26. In particular, tissue piercing members 32 may be, for example, substantially triangularly-shaped with two sides thereof being separated from a respective body tissue contact portion 26, and one side remaining pivotally secured thereto such that tissue piercing member 32 may be pivoted in such a way so as to extend a point portion 34 out from the respective body tissue contact portion 26 and into an operating orientation.

In some embodiments of the present invention, body tissue contact portions 26 may have a roughened outer surface texture in order to assist in grasping the soft tissue against which such body tissue contact portions 26 operably abut. As illustrated in FIGS. 1-3, first and second prongs 20, 22 preferably extend perpendicularly from main body portion 12. First and second prongs 20, 22, however, may instead extend somewhat non-perpendicularly from main body portion 12, such as in compression or distraction configurations, wherein each of first and second prongs 20, 22 form either a slightly acute or slightly obtuse angle with respect to main body portion 12.

A further aspect of the present invention is illustrated in FIG. 4, wherein apertures 42 are disposed in main body portion 112 of affixation device 110. Preferably, correspondingly-configured suture anchoring mechanisms 50 may be selectively and retainably disposed in respective apertures 42 for providing a location at which to anchor sutures and the like. Suture anchoring mechanisms 50 are preferably threadably received in respective apertures 42, though other means for operably and retainably receiving suture anchoring mechanisms 50 at least partially within apertures 42 are contemplated by the present invention. For example, suture anchoring mechanisms 50 may be press-fit or otherwise inserted into respective apertures 42 so as to provide for secure engagement therewith.

As illustrated in FIGS. 5-7, suture anchoring mechanism 50 preferably includes a threaded sidewall 52 having a pitch that is threadably engageable with respective threaded sidewalls 44 of apertures 42. Such thread pitch is preferably sufficiently steep so as to enable full insertion of suture anchoring mechanism 50 within respective aperture 42 in one to two rotations of suture anchoring mechanism 50.

To facilitate threading rotation of suture anchoring mechanism 50 into a respective aperture 42, one or more actuation receptacles 54, 56 are disposed in second end portion 62 of suture anchoring mechanism 50, with first end portion 60 defining a frusto-conical configuration converging toward first open end 59. Actuation receptacle 54 is preferably configured to receive a hex-drive implement such as a screwdriver, while actuation receptacle 56 is preferably configured to operably receive a custom actuation tool of various designs.

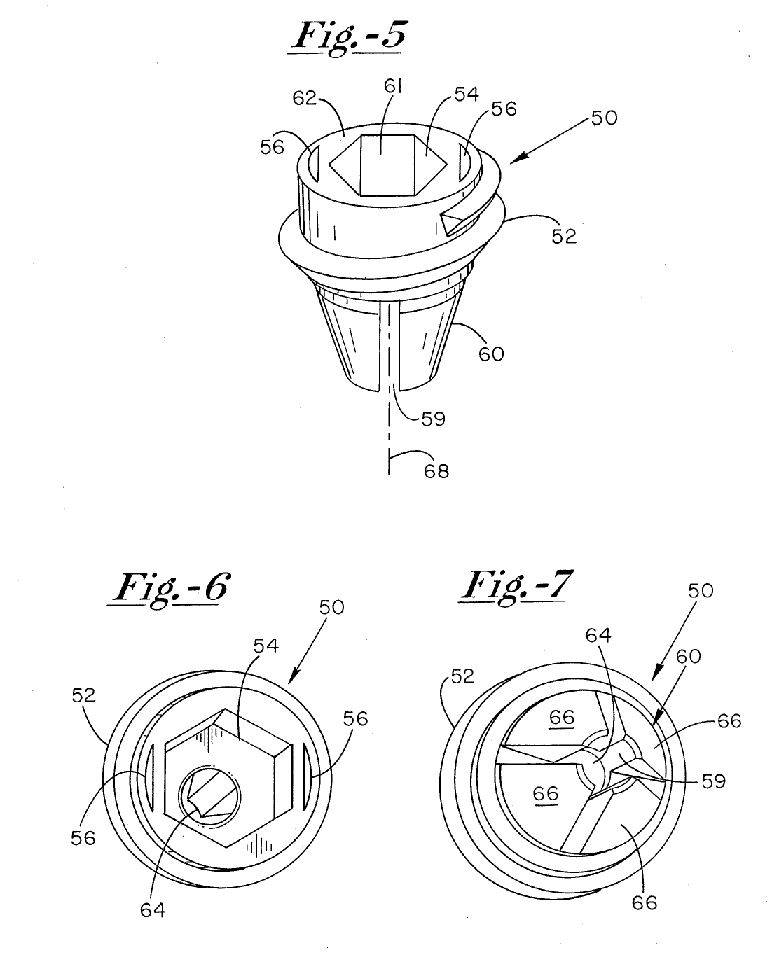

As shown in FIGS. 6 and 7, suture anchoring mechanism 50 preferably includes an open channel 64 extending axially therethrough between first and second opposed open ends 59, 61 thereof. Such open channel 64 is preferably configured to operably receive a portion a suture therethrough. The frusto-conical configuration of first end portion 60 is preferably configured to engage with a tapered internal diameter of the respective aperture 42, such that insertion of suture anchoring mechanism 50 into a respective aperture 42 engages first end portion 60 to a tapered sidewall portion surrounding aperture 42 in main body portion 112. Suture anchoring mechanism 50 is preferably fabricated from a biocompatible polymeric material such as e-PTFE or the like, such that first end portion 60 is relatively flexible. Moreover, first end portion 60 may be formed as separate and distinct spaced-apart quadrants 66 that are compressible toward one another upon engagement of first end portion 60 to the tapered sidewall surrounding aperture 42. Through such a flexibility characteristic, insertion of suture anchoring mechanism 50 into a respective aperture 42 causes the distinct quadrants 66 of first end portion 62 to deflect inwardly toward a central axis 68 of suture anchoring mechanism 50 to thereby lockingly engage the suture at a fixed position within open channel 64.

Through the deflection characteristic described above, suture anchoring mechanism 50 is capable of selectively lockingly engaging upon a suture, a portion of which extends through open channel 64. Since suture anchoring mechanisms 50 may be operably engaged at least partially within respective apertures 42 in main body portion 112, affixation device 110 may act as a secure body to which sutures connecting tissue portions together may be operably anchored. For example, a first tissue portion, such as a bone chip, may be desired to be secured to a second tissue portion, such as a bone. A bore may be drilled through the first tissue portion so that a suture or other tie device may be threaded therethrough and connected to the second tissue portion. The remaining free end of the suture may then be threaded through a respective aperture 42 of main body portion 12 and into engaging relationship with suture anchoring mechanism 50, such as through open channel 64 thereof. Affixation device 110 may then be installed at the first body portion adjacent to, and preferably superimposed over the bore extending therethrough, such that suture anchoring mechanism 50 may be inserted into a respective aperture 42 to lockingly engage the suture to suture anchoring mechanism 50 in a taut condition. Such a mechanism and method provides for a efficient and simple anchoring of sutures securing first and second tissue portions to one another. Moreover, the method and mechanism described above may further act to save soft tissue surrounding, or part of, the first body portion by incorporating a non-planar contour for first surface 113 of affixation device 110, with such non-planar contour providing the tissue-saving benefit described above.

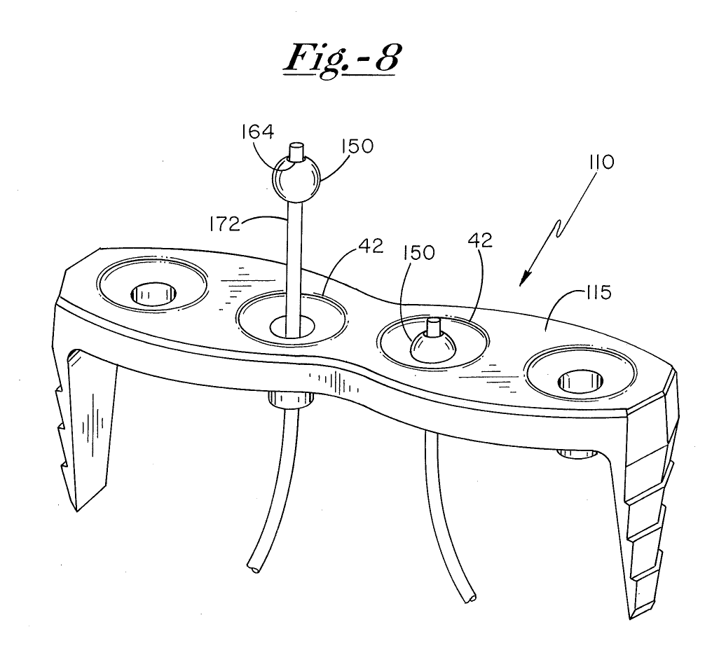

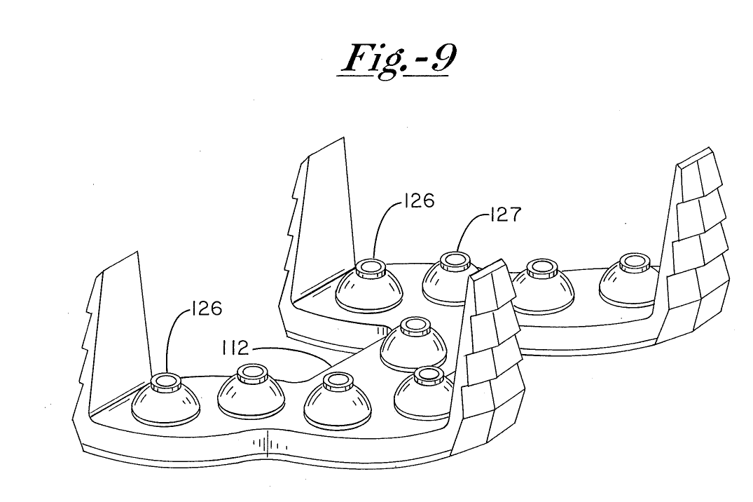

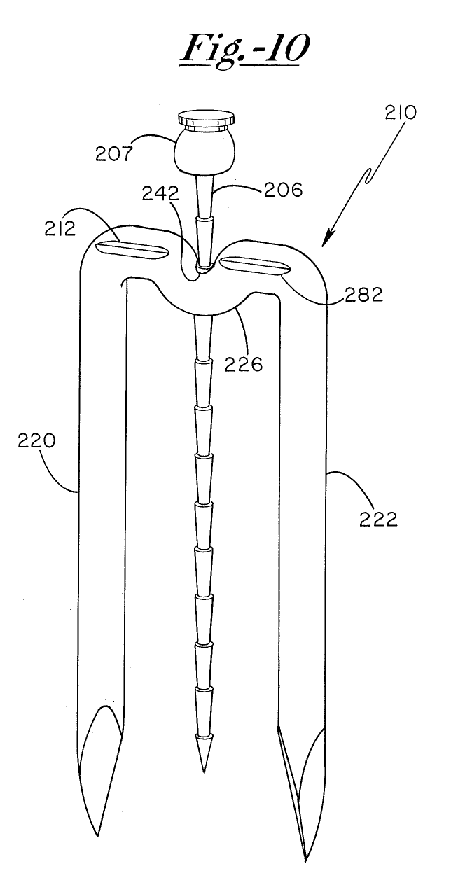

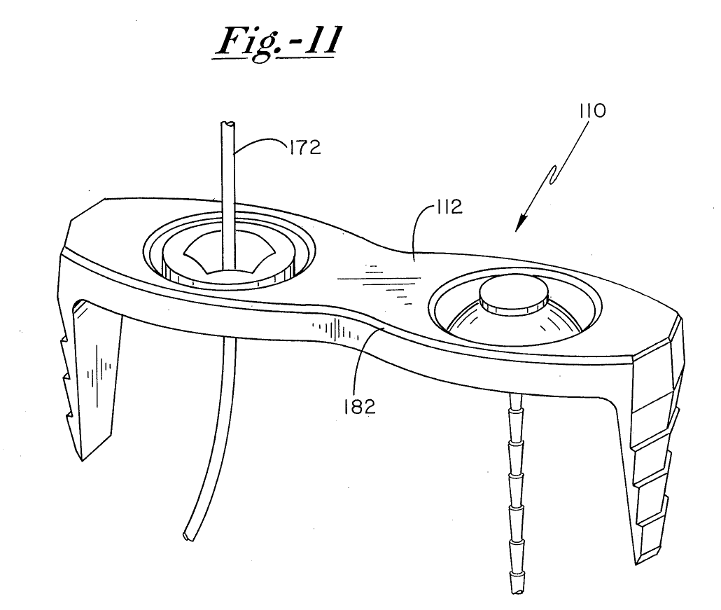

As shown in FIG. 8, suture anchoring mechanism 150 may instead take the form of a sphere having an axial channel 164 bored therethrough for operably receiving suture 172 therein. Open channel 164 may have an interior configuration that restricts motion of suture 172 therethrough to only a single direction, such that engagement of suture anchoring mechanism 150 in a respective aperture 42 lockingly engages suture 172 in place within suture anchoring mechanism 150. In other embodiments, suture anchoring mechanism 150 may deform upon engagement within a respective aperture 42 so as to crimp the radial wall at least partially surrounding open channel 164 upon suture 172, thereby locking suture 172 in place within channel 164.

A vast array of configurations for suture anchoring mechanism 50, 150 are contemplated as being useful in the apparatus of the present invention. In particular, suture anchoring mechanism 50 may be of any design that enables selective locking engagement upon suture 172. For example, suture anchoring mechanism 50 may embody a clip that selectively engages suture 172, and may be operably disposed upon second surface 115 about a respective aperture 42 to thereby fixedly secure suture 172 to affixation device 110.

With reference to FIGS. 4 and 9, body tissue contact portions 126 may be shaped as non-hemispherical protrusions extending from main body portion 112. In particular, respective end sections 127 of body tissue contact portions 126 are preferably configured to maximize the surface area footprint that each body tissue contact portion 126 places upon the impacted soft body tissue.

In preferred embodiments, apertures 42 extend through main body portion 12 and through respective body tissue contact portions 126. As such, suture 172 is preferably threaded through a respective aperture extending axially through a respective body tissue contact portion 126. Moreover, apertures 42 may be provided at portions of main body portion 112 such that some of such apertures 42 axially align with body tissue contact portions 126, while other apertures 42 do not so axially align.

First and second prongs 120, 122 may be serrated so as to enhance the frictional engagement of affixation device 110 into the first body tissue. To further secure the affixation device to the first body tissue, a pin 206 may be inserted through a respective aperture 242 in main body portion 212, and into the first body tissue. In preferred embodiments, pin 206 is serrated to further assist in frictionally engaging with the first body tissue. To assist in holding affixation device 210 to the first body tissue, pin 206 preferably includes a head portion 207 that engages with main body portion 212 upon fully insertion thereof.

Main body portion 212 may further include a lip portion 282 extending outwardly from main body portion 212 along a plane substantially perpendicular to first and second prongs 220, 222. Such a lip portion is useful in providing a grasping surface for an affixation device removal tool, such as a wrench or the like.

As stated above, affixation device 10 is configured such that first and second prongs 20, 22 penetrate a first body tissue so as to secure affixation device 10 thereto. Moreover, body tissue contact portions 26 are provided to maintain non-contacting portions 28 in an installed position that does not negatively impact soft body tissue at the affixation device implantation site. For example, affixation device 10 may be utilized by being secured to a bone, wherein soft tissue surrounds such bone. Installation of affixation device 10 results in first and second prongs 20, 22 penetrating through the soft first body tissue, and into a second body tissue, which in this case is the bone. Body tissue contact portions 26 then come into contact with the soft first body tissue, and prevent further insertion of affixation device 10, so as to provide non-contacting portion 28 of main body portion 12. Such non-contacting portions 28 act to increase the amount of soft tissue left unharmed through the implantation procedure of affixation device 10.

The affixation device of the present invention may be applicable in a large number of surgical procedures. In general, such procedures include grafting soft tissue to bone, bracing bone fractures, and securing bone fragments to bone. Specific examples of surgical procedures in which the affixation device of the present invention may be useful include ligament stapling, tendon grafting, bone fractures, and bone fusion procedures.

Affixation device 10 is preferably fabricated from one or more biodegradable materials, such as stainless steel, titanium, and the like. Other materials, however, may be utilized as being necessary per application.

The invention has been described herein in considerable detail in order to comply with the patent statutes, and to provide those skilled in the art with the information needed to apply the novel principles and to construct and use embodiments of the invention as required. However, it is to be understood that the invention can be carried out by specifically different devices and that various modifications can be accomplished without departing from the scope of the invention itself.

1. A method for affixing a first body tissue portion to a second body tissue portion in vivo, said method comprising:
(a) providing an affixation device having:
(i) a main body portion having a first surface, said first surface having a non-planar contour forming one or more body tissue contact portions and one or more non-contacting portions, said body tissue contact portions having an effective tissue contact surface area that is sufficiently large to facilitate affixation of the first body tissue portion to the second body tissue portion;
(ii) one or more prongs integrally extending from opposed ends of said main body portion; and
(b) installing said affixation device by inserting said one or more prongs through the first body tissue portion and into the second body tissue portion to an extent sufficient to cause said one or more body tissue contact portions to contact the first body tissue portion, and to retainably press at least a portion of the first body tissue portion into intimate contact with the second body tissue portion, said insertion of said one or more prongs into the second body tissue portion securely anchors the first body tissue portion to the second body tissue portion in a manner such that said non-contacting portions remain out of bearing contact with the first body tissue portion.

2. A method as in claim 1 wherein said first body tissue portion is a tendon.

3. A method as in claim 1 wherein said second body tissue portion is a bone.

4. A method as in claim 1 wherein said main body portion includes one or more apertures extending therethrough.

5. A method as in claim 4, including inserting a pin through a respective one of said one or more apertures, and through said first body tissue portion and into said second body tissue portion to an extent such that a head portion of said pin engages said main body portion.

6. A method for securing a first body tissue portion to a second body tissue portion in vivo, said method comprising:
(a) providing an affixation device having:
(i) a main body portion having a first surface and a generally opposed second surface, and one or more apertures extending therethrough between said first and second surfaces, said first surface having a non-planar contour forming one or more body tissue contact portions and one or more non-contacting portions, said body tissue contact portions having a total effective tissue contact surface area that is sufficiently large to facilitate securement of the first body tissue portion to the second body tissue portion while said non-contacting portions remain out of bearing contact with the first body tissue portion;
(ii) one or more prongs extending integrally from opposed ends of said main body portion;
(iii) a suture anchoring mechanism;
(b) securing a first end of an elongated suture to said second body portion;
(c) passing a second end of said suture through a respective one of said one or more apertures, and into engaging relationship with said suture anchoring mechanism;
(d) installing said affixation device by inserting said one or more prongs through the first body tissue portion and into the second body tissue portion to an extent sufficient to cause said one or more body tissue contact portions to contact the first body tissue portion and to retainably press at least a portion of the first body tissue portion into intimate contact with the second body tissue portion; and
(e) positioning said suture anchoring mechanism in engaging relationship with said main body portion.

7. A method as in claim 6 wherein positioning said suture anchoring mechanism in engaging relationship with said main body portion lockingly engages said suture to said suture anchoring mechanism.

8. A method as in claim 6, including positioning said suture anchoring mechanism at least partially within said respective aperture.

9. A method as in claim 6 wherein said suture anchoring mechanism is threadably engaged in said respective aperture.

10. A medical affixation device as in claim 6 wherein said suture anchoring mechanism includes an open channel extending therethrough between first and second open ends of said suture anchoring mechanism.

11. A medical affixation device as in claim 10 wherein said suture anchoring mechanism includes a flexible first end portion at least partially surrounding said first open end.

12. A medical affixation device as in claim 11 wherein said flexible first end portion has a frusto-conical configuration converging towards said first open end.

13. A medical affixation device as in claim 11 wherein said one or more apertures are tapered, such that insertion of said suture anchoring mechanism into said respective aperture causes said flexible first end portion to deflect inwardly toward a central axis of said open channel, thereby lockingly engaging said suture to said suture anchoring mechanism.

14. A medical affixation device as in claim 10, including an actuation receptacle disposed in a second end portion of said suture anchoring mechanism.
